(19) United States
(12) Patent Application Publication
(10) Pub. No.: US 2020/0397686 A1
(43) Pub. Date: Dec. 24, 2020

(54) Methods of Reducing Burning or Stinging Sensation Attributed to Certain Compositions

(71) Applicant: Monell Chemical Senses Center, Philadelphia, PA (US)

(72) Inventors: Mehmet Hakan Ozdener, Springfield, PA (US); Nancy Ellen Rawson, Philadelphia, PA (US)

(21) Appl. No.: 16/979,210

(22) PCT Filed: Mar. 11, 2019
(86) PCT No.: PCT/US2019/021651

§ 371 Date: Sep. 9, 2020

Related U.S. Application Data
(60) Provisional application No. 62/641,481, filed on Mar. 12, 2018.

(51) Int. Cl. A61K 8/60; A61Q 11/00

(52) U.S. Cl. CPC A61K 8/602; A61Q 11/00

(57) ABSTRACT

A method for inhibiting, reducing or eliminating a stinging or burning sensation in the oral cavity caused by exposure to or ingestion of composition that induces the sensation, comprising contacting the oral cavity with an effective amount of at least one sophorolipid before or simultaneously with exposure to the composition.

### CROSS-REFERENCE TO RELATED APPLICATIONS

This application claims the benefit of the priority of U.S. Provisional Patent Application No. 62/641,481, filed Mar. 12, 2018, which application is incorporated herein by reference.

### BACKGROUND OF THE INVENTION

Oral care products have been used worldwide by people for many years for the prevention of bad breath and for the elimination of bacteria and other oral microorganisms that are responsible not only for bad breath, but also tooth decay, plaque, and gum diseases, such as gingivitis and periodontitis. However, the oral health care products have several drawbacks, including an unpleasant aftertaste and, most often, a stinging, burning sensation. Such sensations limit consumer acceptance of oral care products. Current oral care products have dealt with metallic or astringent aftertaste (bad/noxious taste) drawbacks by devising flavor/sweetener formulae to mask the unpleasant taste. This flavor masking has been traditionally accomplished by addition of strong flavors. Unfortunately, high concentrations of masking flavors may irritate the user, and cause the user to have additional unpleasant taste sensations in the oral cavity.

One significant component of oral care products is a surfactant, for example, sodium lauryl sulfate or sodium N-lauroyl sarcosinate. Unfortunately, most toothpaste surfactants have undesirable properties. They taste bad and may irritate oral mucosa. These common surfactants cause inflammation, thus facilitating infection by pathogenic microorganisms and may also aggravate tooth sensitivity and tooth staining in susceptible individuals.

Other ingredients of oral health care products, while adding therapeutic or cosmetic benefit to the product, also result in causing or enhancing unpleasant taste/flavor. Such undesirable properties are particularly important for those who receive oral irradiation and/or chemotherapy and with acute mucositis, dry mouth, loss of taste, etc.

Prior attempts have been made to reduce, or in some cases, enhance, the taste sensations attributed to one or more of the five basic tastes (i.e., bitter, salty, sweet, sour, and umami) in such compositions. See, e.g., References 1-7 identified below. However, there appears to be another unknown mechanism triggered by these oral care compositions (as well as in other orally administered pharmaceutical compositions) that causes a burning or stinging sensation in the oral cavity. Treatment or inhibition of this burning sensation has yet to be addressed. Thus, the active pharmaceutical ingredients and/or inactive ingredients in medicines and over the counter (OTC) compositions producing the burning sensations in the oral cavity remain aversive to children and adults, and such sensations affect compliance with oral care or pharmaceutical treatments negatively.

There remains a need in the art for compositions useful for elimination of certain unpleasant burning and stinging sensations in the oral cavity caused by such oral care and other compositions.

### SUMMARY OF THE INVENTION

In one aspect, a method for inhibiting, reducing or eliminating a stinging or burning sensation in the oral cavity caused by exposure to or ingestion of a composition that induces the sensation, comprises contacting the oral cavity with an effective amount of at least one sophorolipid before or simultaneously with exposure to said composition.

In one embodiment, the method involves adding the sophorolipid to the composition as an additional ingredient. In one embodiment, the sophorolipid is produced using the fatty acid, oleic acid.

In another aspect, an oral care composition is provided which comprises an amount of a sophorolipid sufficient to reduce, inhibit or eliminate a stinging or burning sensation. In still other aspects, oral care compositions are disclosed that comprise a sophorolipid as the sole or primary surfactant, thus eliminating or replacing the common irritating surfactants, sodium lauryl sulfate or sodium N-lauroyl sarcosinate.

Other aspects and advantages of the invention will be readily apparent from the following detailed description of the invention.

### BRIEF DESCRIPTION OF THE DRAWINGS

FIG. 1 is a graph that illustrates that a sophorolipid, having an oleic acid fatty acid tail, when added to mouthwash blocks or inhibits the burning sensation taste produced by use of LISTERINE® mouthwash. As discussed in Example 1 below, six subjects were instructed to rinse their mouths with either control (mouthwash with no sophorolipid) or the treated mouthwash (with 10 μM to 100 μM of the sophorolipid). Subjects reported the results as perceived burning sensation. Five of six subjects reported reductions of at least 50% in perceived burn produced by use of the treated mouthwash vs. use of the control.

### DETAILED DESCRIPTION OF THE INVENTION

Methods and compositions are described herein for inhibiting, reducing or eliminating a stinging or burning sensation in the oral cavity caused by exposure to or ingestion of composition that induces that sensation. While the mechanism for the induction of a burning or stinging sensation in the mouth caused by certain medications or oral care products, and other products, is unknown, the inventors have discovered that contacting the oral cavity with an effective amount of at least one sophorolipid, before or simultaneously with, exposure to the composition can cause a significant reduction in the burning sensation perceived by a human subject.

In one embodiment, the composition causing the burning/stinging sensation is an oral care composition as defined below. In another embodiment, the burning/stinging sensation is caused by an active or accessory ingredient or a combination of ingredients in an oral care composition. In another embodiment, the burning/stinging sensation is caused by an active or accessory ingredient or a combination of ingredients in a pharmaceutical composition. In other embodiments, the burning/stinging sensation is caused by an ingredient or combination of ingredients in an ingestible composition.

As evidenced in the Example below, the inventors demonstrated that sophorolipid (SLs) reduced/eliminated the burning/stinging sensation caused after mouthwash use. In one embodiment, the addition of a sophorolipid in an oral care product is likely to improve the use/acceptance of oral care products. It is an object of the present invention to provide an oral care composition which is consumer preferred. A further object of the present invention is to provide compositions which deliver a variety of benefits to the mouth, such as those described above, with improved taste.

### I. Definitions and Components of the Methods and Compositions

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

A sophorolipid (SL) is a surface-active glycolipid compound of microbial origin which consists of a hydrophilic carbohydrate head, sophorose (2-O-β-D-glucopyranosyl-β-D glucopyranose) attached through a glycosidic linkage to a hydrophobic fatty acid tail of 14 to 24 carbon atoms. The hydrophilic portion, sophorose, is a glucose di-saccharide with an unusual β-1,2 bond and can be acetylated on the 6′- and/or 6″-positions. The specific location of those chemical bonds is dependent on the microbial strain used to produce the SLs, including but not limited to strains of the yeast Candida bombicola. The hydrophobic tail, i.e., the lipid component, is in the embodiment exemplified below an 18-carbon chain fatty acid, having one unsaturated bond, produced using oleic acid. Other fatty acids may be used in the production of sophorolipids useful herein and may have one or two saturated bonds. In still other embodiments, the structural conformation of sophorolipid produced by C. bombicola from glucose and palmitic acid, oleic acid or stearic acid is as a lactone where the carboxylic acid group of the fatty acid is esterified to the disaccharide ring at carbon 4. See the discussion and references cited in US Patent Application Publication US2017/0189533, incorporated by reference herein. In one embodiment, the sophorolipid is produced using the fatty acid, oleic acid. In another embodiment, the sophorolipid is produced using the fatty acid, palmitic acid. In yet another embodiment, the sophorolipid is produced using the fatty acid, stearic acid. In the Example below, the sophorolipid used has an oleic acid tail. Alternatively, suitable sophorolipids may be prepared as described herein or purchased or obtained from publicly available sources.

SL are produced by miicroorganisms (Candida batistae CBS 8550, Rhodotorula bogoriensis, Candida floricola TM1502, Candida riodocensis, Candida rugosa, Candida kuoi, Candida stellate, Candida tropicalis, Cryptococcus sp. VITGBN2, Cyberlindnera samutprakarnensis JP52, Pichia anomala PY1, Rhodotorula muciliginosa, Candida bombicola, Candida apicola, Torulopsis gropengiesseri, Torulopsis petrophilum and Wickerhamiella domercqiae Y2A.⁸

By “effective amount” of the sophorolipid as used herein is meant an amount sufficient to reduce or inhibit the perception of a burning or stinging sensation associated with an oral care composition or pharmaceutical composition, or other ingestible composition producing such a sensation by a significant percentage. In one embodiment, the effective amount ranges from 0.1 μM sophorolipid up to 1×10⁵ μM sophorolipid, depending upon the composition to which the sophorolipid is added. In another embodiment, the effective amount of a sophorolipid in a composition is a range of about 1×10⁴ μM to about 1×10⁵ μM. For purposes of a sophorolipid applied to the oral cavity before or after the subject contacts the composition, the effective amount ranges from 10 μM sophorolipid up to 1×10⁵ μM sophorolipid, in a suitable physiologically or pharmaceutically-acceptable carrier. As one example, the protocol with the LISTERINE mouthwash below demonstrated an effective amount in a mouthwash to be about 10 μM to about 100 μM in mouthwash. The effective amount will vary depending upon whether the sophorolipid is applied before, with or after the composition, and in what carriers. An appropriate effective amount may be determined by one of ordinary skill in the art using only routine experimentation. As used herein, the term “significant percentage” may refer to about 10%, about 15%, about 20%, about 25%, about 30%, about 5%, about 40%, about 45%, about 50%, about 55%, about 60%, about 65%, about 70%, about 75%, about 80%, about 85%, about 90%, about 95%, about 99% or up to 100%.

By physiologically or pharmaceutically-acceptable carrier as used herein is meant include any and all solvents, dispersion media, coatings, antibacterial and antifungal agents, isotonic and absorption delaying agents, adjuvants, surfactants, and the like, compatible with administration to humans. In one embodiment, the diluent is saline or buffered saline. Still other carriers include those identified in texts such as Remington's Pharmaceutical Sciences, 17th edit., 1985 Gennaro, AR eds., Mack Publishing Co, Easton Pa.; and Handbook of Pharmaceutical Excipients, 6th edit., 2009 Rowe R C et al, eds, Pharmaceutical Press, incorporated by reference herein.

In some embodiments, the method of reducing the burning/stinging sensation in a composition comprises incorporating into the composition at least one sophorolipid disclosed herein sufficient to permit replacement of all or a percentage of the other surfactant(s) present in the composition. In one embodiment, an effective amount of a sophorolipid replaces between 25 to 75% of surfactants in an oral care/pharmaceutical/ingestible product that produces a burning sensation, e.g., a mouthwash. In another embodiment, an effective amount of a sophorolipid replaces at least about 5% to about 100% of surfactants in the product. In another embodiment, an effective amount of a sophorolipid replaces at least 1%, 5%, 10%, 15%, 20%, 25%, 30%, 35%, 40%, 45%, 50%, 55%, 60%, 65%, 70%, 75%, 80%, 85%, 90%, 95% or up to 100% including whole and fractional percentages between each number indicated herein.

“Oral health care product” as used herein refers to a product which in the ordinary course of usage is not intentionally swallowed for purposes of systemic administration of therapeutic agents. The oral care product is rather retained in the oral cavity for a time sufficient to contact substantially all the dental surfaces and/or oral tissues for purposes of oral activity. Such products include but are not limited to mouthwashes, toothpaste, gels and dentifrice.

The term “pharmaceutical composition” refers to any OTC or prescription medicine that is taken orally and produces a burning/stinging sensation in the oral cavity or mucosa of the throat.

By “mammalian subject” is meant primarily a human, but also domestic animals, e.g., dogs, cats; and livestock, such as cattle, pigs, etc.; common laboratory mammals, such as primates, rabbits, and rodents; and pest or wild animals, such as deer, rodents, rabbits, squirrels, etc.

By “providing a burning or stinging sensation” is meant that the composition without sophorolipid added creates a burning or stinging sensation that may be unrelated to the taste (e.g., bitter, sweet, sour, salty, umami) normally mediated by oral taste cells. This sensation may be detected by non-taste, e.g., epithelial, cells or taste-like cells within the oral cavity, as well as by taste cells

By “inhibiting or reducing a burning or stinging sensation” is meant that the method or composition described herein can decrease the sensation in the oral cavity when compared to the sensation achieved by the composition which is not associated with a sophorolipid. In one embodiment, the method described herein and/or the use of a composition to which a sophorolipid is added as described herein, reduces the burning or stinging sensation of the composition by up to 25% compared to the original composition without a sophorolipid. In another embodiment, the method or composition inhibits the burning/stinging sensation by up to 50%. In still other embodiments, the methods or compositions described herein reduce or inhibit the burning/stinging sensation by at least 5%, 10%, 15%, 20%, 25%, 30%, 35%, 40%, 45%, 50%, 55%, 60%, 65%, 70%, 75%, 80%, 85%, 90%, 95% or up to 100% including whole and fractional percentages between each number indicated herein.

“Taste cell”, as used herein, refers to a mammalian sensory cell found in taste buds in the oral cavity of mammals, including circumvallate papillae and foliate papillae.

These cells can be identified by their typical appearance under light and electron microscopy. They can also be identified by oral location along with expression of taste specific proteins.

A “taste-like cell” as used herein refers to any cell outside of the oral cavity that expresses a known taste signaling protein. Such cells include solitary chemosensory cells in the lungs and nose, intestinal endocrine cells, pancreatic endocrine cells and others.

“Non-taste cell”, as used herein refers to other cells within the oral cavity including the epithelial cells lining the inside of the lips, cheeks, tonsils, and throat.

By “physiological or behavioral assay for burning or stinging sensation” is meant a known assay relying on physiological or behavioral reactions in response to increased or decreased burning or stinging sensation of a compound applied to the oral cavity of a mammalian test animal or human. Examples of known assays include primarily sip and spit taste assays. See, e.g., Delwiche et al, Food Quality and Preference, 1996 March; 7(3-4)293-7. Other suitable assays are known to those of skill in the art and may be applied in the methods described below.

The terms “a” or “an” refers to one or more, for example, “an assay” is understood to represent one or more assays. As such, the terms “a” (or “an”), “one or more,” and “at least one” are used interchangeably herein.

The term “about” as used herein when referring to a measurable value such as an amount, a temporal duration, and the like, is meant to encompass variations of up to ±10% from the specified value; as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.”

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

### II. Methods and Compositions

In one aspect, a method for inhibiting, reducing or eliminating a stinging or burning sensation in the oral cavity caused by exposure to or ingestion of composition that induces the sensation, comprising involves contacting the oral cavity with an effective amount of at least one sophorolipid substantially simultaneously with the composition causing the sensation. In one embodiment, an effective amount of the selected sophorolipid is added to the composition to reduce the burning/stinging sensation when the subject uses the composition. For example, where the composition is a mouthwash, such as LISTERINE brand mouthwash, a final concentration of about 1×10 to about 1×10² μM sophorolipid is added to the mouthwash. As used herein, the term “final concentration” refers to the concentration of a sophorolipid in the composition, e.g., mouthwash. Where the composition is another oral care liquid, the amount of sophorolipid added is proportional to the degree of burning sensation ascribed by the subject.

In one embodiment, a method for inhibiting, reducing or eliminating a stinging or burning sensation in the oral cavity caused by exposure to or ingestion of composition that induces the sensation, comprising involves contacting the oral cavity with an effective amount of at least one sophorolipid before exposure or after exposure to the indicated composition. In such an embodiment, the sophorolipid may be provided in a separate liquid solution or rinse in a pharmaceutically acceptable carrier or diluent for use by the subject before or after exposure to the composition, e.g., mouthwash. The subject would rinse immediately before using the mouthwash or immediately afterwards to reduce the burning/stinging sensation.

In yet another aspect, the sophorolipid may be added to a pharmaceutical composition or any other composition producing a burning/stinging sensation or used as a pre- or post-rinse in the same manner.

In one specific embodiment, a final concentration of about 1×10⁴ μM to about 1×10⁵ μM of a sophorolipid produced by use of oleic acid is added to mouthwash to reduce the burning/stinging sensation, as shown in Example 1 below. It is understood that other mouthwashes than LISTERINE brand mouthwash may be used in a similar manner.

In still other embodiments, the method involves adding a sophorolipid to a liquid, gel, semi-solid, emulsion or solid form of an oral care product (or other product) during the preparation thereof. In yet other embodiments, the sophorolipid is added to any of these compositions to replace other ingredients, such as surfactants.

It is further anticipated that this use of a sophorolipid may reduce the burning/stinging sensation of other ingestible compositions, including those for oral care, pharmaceutical preparations or ingestible lozenges and other products.

Thus, as another aspect, is a composition comprising an effective amount of a sophorolipid or mixture thereof, wherein the sophorolipid is employed to replace other ingredients in the original composition.

In still another aspect is a method for alleviating discomfort in subjects receiving oral irradiation and/or chemotherapy by treating said subjects with oral care products or pharmaceutical preparations containing a desired amount of a sophorolipid, as described above.

In still another aspect is a method for alleviating discomfort in subjects having acute mucositis, dry mouth, or loss of taste, among other oral cavity disorders by treating said subjects with oral care products or pharmaceutical preparations containing a desired amount of a sophorolipid, as described above.

The following example is illustrative of the claimed invention.

### Example 1: Reduction of Burning Sensation in Oral Care Product

The following experiment was performed to demonstrate that a sophorolipid, e.g., 1×10⁴ μM to 1×10⁵ μM which is the lactone formed by the association of sophorose with an oleic acid tail, would block, inhibit, or reduce the burning sensation in the mouth produced by use of LISTERINE® mouthwash (Johnson & Johnson Consumer, Inc.). Six subjects were involved in the study.

The sophorolipid used in this experiment was obtained from the US Department of Agriculture. Alternatively, however, a suitable sophorolipid may be produced as described in US2017/0189533 at the 10-L bench-top scale from C. bombicola ATCC 22214 (American Type Culture Collection, Manassas, Va.) grown on glucose and oleic acid (Sigma Chemical, St. Louis, Mo.). The basal Candida Growth Media (CGM) (10% glucose, 1% yeast extract, 0.1% urea) was prepared, sterilized by autoclave, and the temperature was equilibrated to 26° C. Fatty acid was added to the CGM as the lipid co-substrate, either as non-miscible liquid at a final concentration of 2% by weight. The fermentations were conducted at a temperature of 26° C., an agitation rate set at 700 rpm, an air-flow rate of 2 L of air/min, and no pH control. After 2 days, an additional 7.5% (w/v) of dry glucose and 2% (w/v) fatty acid were added to the fermentations and the fermentations allowed to proceed to 5 days when an additional 0.5% (w/v) of fatty acid was added. The fermentations then continued for an additional 2 days (total duration of the fermentation was 7 days). Sophorolipid isolation and recovery was accomplished by lyophilizing the entire culture to dryness for −2 days. The dried residue was divided and placed into three 6-L Erlenmeyer flasks. Each fraction was extracted with excess ethyl acetate at room temperature for two days. The extract was filtered through Whatman No. 2 filter paper to remove any insoluble material (e.g., residual yeast cells). The remaining Solids were washed two additional times with ethyl acetate to maximize recovery. The ethyl acetate fractions were combined, concentrated by evaporation, and precipitated into 1 L aliquots of hexane to obtain the pure SL. The pure SL was recovered from the hexane by filtration (described above) and vacuum-dried in a desiccator to obtain a fine white to off-white colored powder. Yield was determined gravimetrically.

A final concentration of 1×10⁴ μM to 1×10⁵ μM of the above noted sophorolipid was added to the mouthwash. The mouthwash with no sophorolipid added was the control. The subjects were told to rinse their mouths with either control or the treated mouthwash and hold it in their mouths for 10-60 seconds before spitting. Subjects were told to report the results as perceived burning sensation.

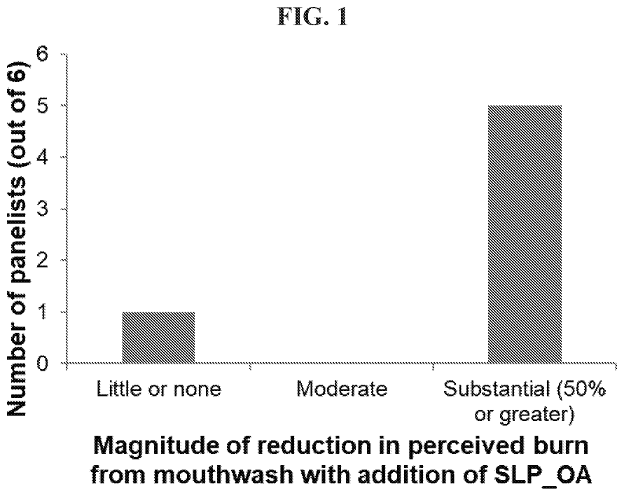

As reported in the graph of FIG. 1, five of six subjects reported reductions of at least 50% in perceived burn produced by use of the untreated mouthwash vs. by use of the treated mouthwash.

All publications and documents cited in this specification are incorporated herein by reference herein. Some of these documents are incorporated to provide instruction on methods or components available to one of skill in the art, which may be used according to the teachings of this specification. While the invention has been described with reference to particular embodiments, it will be appreciated that modifications can be made without departing from the spirit of the invention. Such modifications are intended to fall within the scope of the appended claims.

### REFERENCES

1. International Patent Application No. WO2017/117049 for Compositions Containing a Bitter Tastant and at Least One Sophorolipid, and Methods of Reducing Bitter Taste Attributed to a Bitter Tastant in an Edible Composition, published Jul. 6, 2017
2. US Patent Application Publication No. US2017/0189533 for Compositions Containing a Bitter Tastant and at Least One Sophorolipid, and Methods of Reducing Bitter Taste Attributed to a Bitter Tastant in an Edible Composition, published Jul. 6, 2017
3. U.S. Pat. No. 6,121,315, for Oral Compositions Containing a Zinc Compound, published Sep. 19, 2002
4. US Patent Application Publication No. US2014/0287122, for Use of Rubusoside for Reducing or Suppressing Certain Unpleasant Taste Impressions, published Sep. 25, 2014
5. International Patent Application No. WO2012/112141 for Taste Masked Oral Care Compositions, published Aug. 23, 2012
6. International Patent Application No. Wo2008/005548 for Flavors for Oral Compositions, published Jan. 10, 2008
7. Sohi, H. et al, (2004, May) Taste Masking Technologies in Oral Pharmaceuticals: Recent Developments and Approaches, Drug Development and Industrial Pharmacy, 30(5):429-448
8. De Oliveira, M R et al, Review: Sophorolipids A Promising Biosurfactant and it's Applications., Internatl J. Adv Biotechnol. Res. 6(2):161-174 (2015)

1. A method for inhibiting, reducing or eliminating a stinging or burning sensation in the oral cavity caused by exposure to or ingestion of a composition that induces the sensation, comprising contacting the oral cavity with an effective amount of at least one sophorolipid before or simultaneously with exposure to said composition.

2. The method according to claim 1, wherein the composition is an oral care composition.

3. The method according to claim 1, wherein the composition is a pharmaceutical composition.

4. The method according to claim 1, wherein the sophorolipid has an oleic acid tail.

5. The method according to claim 1, wherein the sophorolipid has a palmitic acid tail.

6. The method according to claim 1, wherein the sophorolipid has a stearic acid tail.

7. The method according to claim 1, further comprising adding an effective amount of the sophorolipid to the composition prior to said exposure or ingestion.

8. A composition for use in inhibiting, reducing or eliminating a stinging or burning sensation in the oral cavity caused by exposure to or ingestion of a second composition that induces the sensation, comprising an effective amount of at least one sophorolipid.

9. The composition according to claim 8, wherein the second composition is an oral care composition.

10. The composition according to claim 8, wherein the second composition is a pharmaceutical composition.

11. The composition according to claim 8, wherein the sophorolipid has an oleic acid tail.

12. The composition according to claim 8, wherein the sophorolipid has a palmitic acid tail.

13. The composition according to claim 8, wherein the sophorolipid has a stearic acid tail.

14. The composition according to claim 8, wherein the sophorolipid composition is contacted with the oral cavity before or simultaneously with exposure to the second composition.

15. The composition according to claim 8, wherein the second composition comprises the sophorolipid.

16-23. (canceled)
